(19) United States
(12) Patent Application Publication
(10) Pub. No.: US 2026/0240932 A1
(43) Pub. Date: Aug. 20, 2026

(54) COMPOSITION FOR ENHANCING SKIN BARRIER CONTAINING COMPLEX EXTRACT OF RED GINSENG, CENTELLA ASIATICA, HOUTTUYNIA CORDATA AND LONICERAE FLOS AS ACTIVE INGREDIENTS

(71) Applicant: INDUSTRY-ACADEMIC COOPERATION FOUNDATION DAEGU HAANY UNIVERSITY, Gyeongsan-si (KR)

(72) Inventors: Jin Han PARK, Daegu (KR); Mi Mi Park, Cheongdo-gun (KR)

(73) Assignee: INDUSTRY-ACADEMIC COOPERATION FOUNDATION DAEGU HAANY UNIVERSITY, Gyeongsan-si (KR)

(21) Appl. No.: 19/430,820

(22) Filed: Dec. 23, 2025

(30) Foreign Application Priority Data
Feb. 20, 2025 (KR) 10-2025-0022136

(51) Int. Cl. A61K 36/258; A61K 8/9789; A61K 36/23; A61K 36/355; A61K 36/78; A61P 17/00; A61P 17/18; A61P 29/00; A61Q 19/00

(52) U.S. Cl. CPC A61K 36/258; A61K 8/9789; A61K 36/23; A61K 36/355; A61K 36/78; A61P 17/00; A61P 17/18; A61P 29/00; A61Q 19/00; A61K 2236/333; A61K 2236/51

(57) ABSTRACT

The present disclosure relates to a composition for enhancing a skin barrier, comprising a complex extract of red ginseng, Centella asiatica, Houttuynia cordata, and Lonicerae Flos as active ingredients. The complex extract of red ginseng, Centella asiatica, Houttuynia cordata, and Lonicerae Flos of the present disclosure has no cytotoxicity and has excellent anti-inflammatory and antioxidant effects, and thus may be effectively used as a cosmetic composition or health functional food composition for enhancing a skin barrier and may be effectively used as a cosmetic composition or pharmaceutical composition for improving skin inflammation.

### CROSS-REFERENCE TO RELATED APPLICATIONS

This application claims the priority of Korean Patent Application No. 10-2025-0022136 filed on Feb. 20, 2025, in the Korean Intellectual Property Office, the disclosure of which is incorporated herein by reference.

### STATEMENT REGARDING SPONSORED RESEARCH

The present disclosure is a result of research conducted with the support of “Evaluation of Memory Improvement Efficacy of Food Composition Containing Red Ginseng (Project No. 24-700-044)” project from DANA.
Project Number: 24-700-044
Ordering company: DANA
Project management agency name: Daegu Haany University Industry-Academic Cooperation Foundation
Research business name: Research service business (other business)
Research project name: Evaluation of memory-improving efficacy of food compositions containing red ginseng
Project institution name: Department of Cosmetics, Daegu Haany University
Research Period: Sep. 11 to Nov. 10, 2024

### Background

### Field

The present disclosure relates to a composition for enhancing a skin barrier, including a complex extract of red ginseng, Centella asiatica, Houttuynia cordata, and Lonicerae Flos as active ingredients.

The present disclosure is a result of research conducted with the support of “Evaluation of the memory improvement efficacy of food compositions containing red ginseng (Project No. 24-700-044)” project from DANA.

### Description of the Related Art

The skin is the outermost organ of the human body and performs an essential barrier function of protecting the moisture in the body and preventing external invasion factors (antigens, infectious agents, etc.) from entering the body. The skin has several barriers depending on a barrier function. That is, there are a physical barrier consisting of the stratum corneum and tight junctions, a chemical/biochemical barrier consisting of lipids, acids, lysosomes, and antimicrobial peptides, and an immune barrier against infectious diseases.

The stratum corneum (horny layer) is formed from keratinocytes and consists of differentiated keratinocytes and lipid layers surrounding the keratinocytes. The keratinocytes are characteristic cells in which basal cells continuously proliferating in the lowest layer of the epidermis rise to the skin surface through gradual changes in form and function. After a certain period of time, old keratinocytes fall off from the skin and new keratinocytes take over their function, and thus a repetitive series of changes is called differentiation or keratinization of epidermal cells. This stratum corneum may easily lose its function due to lifestyle factors such as excessive washing or bathing, environmental factors such as dry air pollutants, and endogenous diseases such as atopic skin or aged skin, and actually, due to a further increase in various factors in modern times, parts appealing dry skin symptoms and various disorders caused by the dry skin symptoms are gradually increasing. In addition, as the external environment changes and aging are developed, various changes occur in the skin to damage the skin barrier, thereby causing skin inflammation, dryness, and itchiness.

Accordingly, the present inventors have confirmed that a complex extract prepared by mixing red ginseng, Centella asiatica, Houttuynia cordata, and Lonicerae Flos has no cytotoxicity and has excellent anti-inflammatory and antioxidant effects, and thus may be effectively used as a composition for enhancing a skin barrier or improving skin inflammation, and then completed the present disclosure.

### PRIOR ARTS

### Patent Documents

Korean Patent Registration No. 10-2314736.

### SUMMARY

An object of the present disclosure is to provide a cosmetic composition for enhancing a skin barrier, including a complex extract prepared by mixing red ginseng, Centella asiatica, Houttuynia cordata, and Lonicerae Flos as active ingredients.

Another object of the present disclosure is to provide a health functional food composition for enhancing a skin barrier, including a complex extract prepared by mixing red ginseng, Centella asiatica, Houttuynia cordata, and Lonicerae Flos as active ingredients.

Yet another object of the present disclosure is to provide a cosmetic composition for improving skin inflammation, including a complex extract prepared by mixing red ginseng, Centella asiatica, Houttuynia cordata, and Lonicerae Flos as active ingredients.

Still another object of the present disclosure is to provide a pharmaceutical composition for preventing or treating skin inflammation, including a complex extract prepared by mixing red ginseng, Centella asiatica, Houttuynia cordata, and Lonicerae Flos as active ingredients.

Still another object of the present disclosure is to provide a method for preparing a complex extract.

In order to achieve the object, an aspect of the present disclosure provides a cosmetic composition for enhancing a skin barrier, including a complex extract of red ginseng, Centella asiatica, Houttuynia cordata, and Lonicerae Flos as active ingredients.

Another aspect of the present disclosure provides a health functional food composition for enhancing a skin barrier, including a complex extract prepared by mixing red ginseng, Centella asiatica, Houttuynia cordata, and Lonicerae Flos as active ingredients.

Yet another aspect of the present disclosure provides a cosmetic composition for improving skin inflammation, including a complex extract prepared by mixing red ginseng, Centella asiatica, Houttuynia cordata, and Lonicerae Flos as active ingredients.

Still another aspect of the present disclosure provides a pharmaceutical composition for preventing or treating skin inflammation, including a complex extract prepared by mixing red ginseng, Centella asiatica, Houttuynia cordata, and Lonicerae Flos as active ingredients.

Still another aspect of the present disclosure provides a method for preparing a complex extract including: a) preparing a mixture by mixing red ginseng, Centella asiatica, Houttuynia cordata, and Lonicerae Flos; and b) preparing a complex extract by ultrasonically extracting the mixture.

According to the present disclosure, the complex extract of red ginseng, Centella asiatica, Houttuynia cordata, and Lonicerae Flos has no cytotoxicity and has excellent anti-inflammatory and antioxidant effects, and thus can be effectively used as a cosmetic composition or health functional food composition for enhancing a skin barrier and can be effectively used as a cosmetic composition or pharmaceutical composition for improving skin inflammation.

The effects of the present disclosure are not limited to the aforementioned effects, and other effects, which are not mentioned above, will be apparently understood to a person having ordinary skill in the art from the following description.

The objects to be achieved by the present disclosure, the means for achieving the objects, and the effects of the present disclosure described above do not specify essential features of the claims, and, thus, the scope of the claims is not limited to the disclosure of the present disclosure.

### BRIEF DESCRIPTION OF DRAWINGS

The above and other aspects, features and other advantages of the present disclosure will be more clearly understood from the following detailed description taken in conjunction with the accompanying drawings, in which:

FIG. 1 is a diagram showing results of analyzing electron donating ability of a complex extract according to an exemplary embodiment of the present disclosure;

FIG. 2 is a diagram showing results of analyzing ABTS radical scavenging activity of a complex extract according to an exemplary embodiment of the present disclosure;

FIG. 3 is a diagram showing results of analyzing reducing power of a complex extract according to an exemplary embodiment of the present disclosure;

FIG. 4 is a diagram showing results of analyzing cytotoxicity of a complex extract according to an exemplary embodiment of the present disclosure;

FIG. 5 is a diagram showing results of analyzing NO production inhibition ability of a complex extract according to an exemplary embodiment of the present disclosure;

FIG. 6 is a diagram showing results of analyzing IL-6 production inhibition ability of a complex extract according to an exemplary embodiment of the present disclosure; and

FIG. 7 is a diagram showing results of analyzing TNF production inhibition ability of a complex extract according to an exemplary embodiment of the present disclosure.

### DETAILED DESCRIPTION OF THE EMBODIMENT

Hereinafter, exemplary embodiments of the present disclosure will be described in detail with reference to the accompanying drawings. However, the following exemplary embodiments are presented as examples for the present disclosure, and when it is determined that a detailed description of well-known technologies or configurations known to those skilled in the art may unnecessarily obscure the gist of the present disclosure, the detailed description thereof may be omitted, and the present disclosure is not limited thereto. Various modifications and applications of the present disclosure may be made within the description of claims to be described below and the equivalent scope interpreted therefrom.

In addition, terminologies used in the present disclosure are terminologies used to properly express preferred exemplary embodiments of the present disclosure, which may vary according to a user, an operator's intention, or customs in the art to which the present disclosure pertains. Accordingly, definitions of the terminologies need to be described based on contents throughout this specification. Throughout this specification, unless explicitly described to the contrary, when a certain part “comprises” a certain component, it will be meant to further comprise other components rather than excluding other components.

Throughout this specification, ‘%’ used to indicate the concentration of a specific material is solid/solid (w/w) %, solid/liquid (w/v) %, and liquid/liquid (v/v) %, unless otherwise stated.

In one aspect, the present disclosure provides a cosmetic composition for enhancing a skin barrier, including a complex extract prepared by mixing red ginseng, Centella asiatica, Houttuynia cordata, and Lonicerae Flos as active ingredients.

In an exemplary embodiment of the present disclosure, the complex extract may be prepared by mixing red ginseng, Centella asiatica, Houttuynia cordata, and Lonicerae Flos in a weight ratio of 2 to 4:1 to 3:1 to 3:0.1 to 2, preferably 3:2:2:1, but is not limited thereto.

In an exemplary embodiment of the present disclosure, the complex extract may be extracted with water, lower alcohols having 1 to 4 carbon atoms, or a mixed solvent thereof, preferably ethanol, more preferably 50 to 70% ethanol, and even more preferably 60% ethanol, but is not limited thereto.

In an exemplary embodiment of the present disclosure, the complex extract may be prepared by mixing 20 to 40 parts by weight of red ginseng, 10 to 30 parts by weight of Centella asiatica, 10 to 30 parts by weight of Houttuynia cordata, and 0.1 to 20 parts by weight of Lonicerae Flos, but is not limited thereto.

In an exemplary embodiment of the present disclosure, the complex extract may be prepared by mixing red ginseng, Centella asiatica, Houttuynia cordata, and Lonicerae Flos with an extract solvent in a ratio of 1:1 to 15 (w/v), preferably 1:4 to 9 (w/v), more preferably 1:5 to 7 (w/v), and even more preferably 1:6.25 (w/v), but is not limited thereto.

In an exemplary embodiment of the present disclosure, the complex extract may be prepared by ultrasonic extraction at 30 to 70° C., preferably 40 to 60° C., more preferably 50° C., but is not limited thereto.

In an exemplary embodiment of the present disclosure, the ultrasonic extraction may be performed 1 to 3 times, preferably 2 times, but is not limited thereto.

In an exemplary embodiment of the present disclosure, the complex extract may have an antioxidant effect, but is not limited thereto.

In an exemplary embodiment of the present disclosure, the complex extract may have an anti-inflammatory effect, but is not limited thereto.

The extract according to the present disclosure may be obtained and used by extraction and separation from the nature using extraction and separation methods known in the art. The “extract” as defined in the present disclosure is extracted from the mixture of red ginseng, Centella asiatica, Houttuynia cordata, and Lonicerae Flos using a suitable solvent, and includes, for example, a crude extract, a polar solvent-soluble extract, or a non-polar solvent-soluble extract. As the suitable solvent for extracting the extract from the mixture of red ginseng, Centella asiatica, Houttuynia cordata, and Lonicerae Flos, any pharmaceutically acceptable organic solvent may be used, and water or an organic solvent may be used, but is not limited thereto. For example, various solvents such as purified water, alcohols having carbon atoms 1 to 4 including methanol, ethanol, propanol, isopropanol, butanol, etc., acetone, ether, benzene, chloroform, ethyl acetate, methylene chloride, hexane, cyclohexane, and the like may be used alone or in combination, and preferably, ethanol was used. As the extraction method, any one of methods such as hot water extraction, chilling extraction, reflux cooling extraction, solvent extraction, steam distillation, ultrasonic extraction, elution, and compression may be selected and used. In addition, a desired extract may also be further subjected to a conventional fractionation process or also purified using a conventional purification method.

A preparation method of the extract of the present disclosure is not limited, and any known preparation method may also be used. For example, the extract included in the composition of the present disclosure may be prepared in a powder form with a primary extract extracted by the hot water extraction or solvent extraction method by an additional process such as distillation under reduced pressure and freeze-drying or spray-drying. In addition, fractions may also be obtained by further purifying the primary extract using various chromatography, such as silica gel column chromatography, thin layer chromatography, and high performance liquid chromatography. Accordingly, in the present disclosure, the extract is a concept that includes all extracts, and fractioned and purified products that are obtained in each step of extraction, fractionation, or purification, and dilutions, concentrates, or dried products thereof.

In one aspect, the present disclosure provides a health functional food composition for enhancing a skin barrier, including a complex extract prepared by mixing red ginseng, Centella asiatica, Houttuynia cordata, and Lonicerae Flos as active ingredients.

The “health functional food” used in the present disclosure is the same term as food for special health use (FoSHU), and refers to food with a high medical/care effect, which is processed so as to effectively exhibit bio-regulatory functions in addition to nutrition supply. Here, the “function” refers to adjusting nutrients or to obtaining effects useful for health applications such as physiological actions with respect to the structures and functions of the human body. The health functional food of the present disclosure is able to be prepared by methods which are commonly used in the art, and may be prepared by adding raw materials and ingredients to be commonly added in the art during preparation. In addition, formulations of the health functional food may also be prepared without limitations as long as the formulation is any formulation recognized as food. The health functional food of the present disclosure may be prepared in various types of formulations, and unlike general drugs, the health functional food has an advantage of having no side effects that may occur when taking drugs for a long time by using the food as a raw material, and has excellent portability, so that the health functional food of the present disclosure is able to be taken as a supplement to enhance the effect of enhancing the skin barrier.

The health functional food refers to food that has a more active health maintenance or promotion effect than general foods, and the health supplement food refers to food for the purpose of health supplementation. In some cases, the terms of the health functional food, the health food, and the health supplement food may be used interchangeably.

Specifically, the health functional food refers to food prepared by adding the composition of the present disclosure to food materials such as beverages, teas, spices, gums, confectionery, etc., or prepared in the form of encapsulation, powder, suspension, etc., which brings about a specific health effect when consumed, but has an advantage of having no side effects that may occur when taking drugs for a long time by using the food as a raw material, unlike general drugs.

The health functional food may further include a physiologically acceptable carrier, and the type of carrier is not particularly limited, and any carrier commonly used in the art may be used.

In addition, the health functional food may include an additional ingredient which is commonly used in the food to enhance smell, taste, sight, and the like. For example, the health functional food may include vitamins A, C, D, E, B1, B2, B6, and B12, niacin, biotin, folate, pantothenic acid, and the like. In addition, the health functional food may include minerals such as zinc (Zn), iron (Fe), calcium (Ca), chromium (Cr), magnesium (Mg), manganese (Mn), and copper (Cu); and amino acids such as lysine, tryptophan, cysteine, and valine.

In addition, the health functional food may include food additives, such as preservatives (potassium sorbate, sodium benzoate, salicylic acid, sodium dehydroacetate, etc.), disinfectants (bleaching powder and highly bleaching powder, sodium hypochlorite, etc.), antioxidants (butylhydroxyanisole (BHA), butylhydroxytoluene (BHT), etc.), colorants (tar color, etc.), coloring agents (sodium nitrite, sodium nitrite, etc.), bleach (sodium sulfite), seasoning (MSG sodium glutamate, etc.), sweeteners (dulcin, cyclamate, saccharin, sodium, etc.), flavorings (vanillin, lactones, etc.), expanding agents (alum, D-potassium bitartrate, etc.), reinforcing agents, emulsifying agents, thickening agents, coating agents, gum base agents, foam inhibitors, solvents, ameliorants, etc. The additives may be selected according to a type of food and used in an appropriate amount.

As an example of the health functional food of the present disclosure, a health beverage composition may be used, and in this case, may contain various flavoring agents or natural carbohydrates as additional ingredients, like general beverages. The natural carbohydrates described above may include monosaccharides such as glucose and fructose; disaccharides such as maltose and sucrose; polysaccharides such as dextrin and cyclodextrin; and sugar alcohols such as xylitol, sorbitol, and erythritol. As the sweeteners, natural sweeteners such as thaumatin and stevia extracts; synthetic sweeteners such as saccharin and aspartame, and the like may be used. The proportion of the natural carbohydrates may be generally about 0.01 to 0.04 g, specifically about 0.02 to 0.03 g, per 100 mL of the health beverage composition of the present disclosure.

In addition, the health beverage composition may contain various nutrients, vitamins, electrolytes, flavoring agents, coloring agents, pectic acid and salts thereof, alginate and salts thereof, organic acids, protective colloidal thickeners, pH adjusting agents, stabilizers, preservatives, glycerin, alcohols, carbonating agents, or the like. In addition, the health beverage composition may contain fruit pulps for preparing natural fruit juice, fruit juice beverage or vegetable beverage. These ingredients may be used independently or in combination. The ratio of these additives is not very important, but is generally selected in a range of 0.01 to 0.1 part by weight per 100 parts by weight of the health beverage composition of the present disclosure.

In one aspect, the present disclosure provides a cosmetic composition for improving skin inflammation, including a complex extract prepared by mixing red ginseng, Centella asiatica, Houttuynia cordata, and Lonicerae Flos as active ingredients.

The “cosmetic composition” of the present disclosure may be prepared by including a cosmetically effective amount of the complex extract prepared by mixing red ginseng, Centella asiatica, Houttuynia cordata, and Lonicerae Flos of the present disclosure described above, and a cosmetically acceptable carrier.

As used in the present disclosure, the term “cosmetically effective amount” means an amount enough to achieve a skin whitening effect of the composition of the present disclosure described above.

The appearance of the cosmetic composition contains a cosmetically or dermatologically acceptable medium or base. The appearance of the cosmetic composition may be provided in all formulations suitable for local application, for example, in the forms of solutions, gels, solids, anhydrous paste products, emulsions obtained by dispersing an oil phase in a water phase, suspensions, microemulsions, microcapsules, microgranules or ionic (liposomal) and non-ionic vesicular dispersions, or in the forms of creams, toners, lotions, powders, ointments, sprays or concealer sticks. These compositions may be prepared according to conventional methods in the art. The composition according to the present disclosure may also be used in the form of a foam or in the form of an aerosol composition further containing a compressed propellant.

The cosmetic composition according to an exemplary embodiment of the present disclosure is not particularly limited in its formulation, and may be formulated in cosmetics, such as softening toner, astringent toner, nourishing toner, nourishing cream, massage cream, essence, eye cream, eye essence, cleansing cream, cleansing foam, cleansing water, pack, powder, body lotion, body cream, body oil, and body essence.

When the formulation of the cosmetic composition of the present disclosure is the paste, cream, or gel, as the carrier ingredient, animal oils, vegetable oils, wax, paraffin, starch, tragacanth, cellulose derivatives, polyethylene glycol, silicone, bentonite, silica, talc, zinc oxide or the like may be used.

When the formulation of the cosmetic composition of the present disclosure is the powder or spray, as the carrier ingredient, lactose, talc, silica, aluminum hydroxide, calcium silicate or polyamide powder may be used, and particularly, in the case of the spray, a propellant such as chlorofluorohydrocarbon, propane/butane or dimethyl ether may be additionally included.

When the formulation of the cosmetic composition of the present disclosure is the solution or emulsion, as the carrier ingredient, a solvent, a solubilizing agent or an emulsifying agent may be used. For example, the carrier ingredients include water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or fatty acid ester of sorbitan.

When the formulation of the cosmetic composition of the present disclosure is the suspension, as the carrier ingredient, a liquid diluent such as water, ethanol or propylene glycol, a suspension such as ethoxylated isostearyl alcohol, polyoxyethylene sorbitol ester and polyoxyethylene sorbitan ester, microcrystalline cellulose, aluminum metahydroxide, bentonite, agar, tragacanth, or the like may be used.

When the formulation of the cosmetic composition of the present disclosure is the surfactant-containing cleansing, as the carrier ingredient, aliphatic alcohol sulfate, aliphatic alcohol ether sulfate, sulfosuccinic acid monoester, isethionate, imidazolinium derivatives, methyltaurate, sarcosinate, fatty acid amide ether sulfate, alkylamido betaine, aliphatic alcohol, fatty acid glyceride, fatty acid diethanolamide, vegetable oil, lanolin derivatives, ethoxylated glycerol fatty acid ester, or the like may be used.

The cosmetic composition of the present disclosure may be applied to cosmetics, such as skin, lotion, cream, essence, pack, foundation, color cosmetics, sunscreen, two-way cake, face powder, compact, makeup base, skin cover, eye shadow, lipstick, lip gloss, lip fix, eyebrow pencil, and toner, and cleaning agents, such as shampoo and soap.

The cosmetic composition according to an exemplary embodiment of the present disclosure may further include functional additives and ingredients included in general cosmetic compositions, in addition to the complex extract prepared by mixing red ginseng, Centella asiatica, Houttuynia cordata, and Lonicerae Flos. The functional additives may include ingredients selected from the group consisting of water-soluble vitamins, oil-soluble vitamins, polymer peptides, polymer polysaccharides, sphingolipids, and seaweed extracts.

In addition to the functional additives, the cosmetic composition of the present disclosure may also be mixed with ingredients included in general cosmetic compositions, if necessary. Other mixed ingredients to be included may include oil and fat ingredients, moisturizers, emollients, surfactants, organic and inorganic pigments, organic powders, ultraviolet absorbers, preservatives, disinfectants, antioxidants, plant extracts, pH adjusters, alcohols, pigments, fragrances, blood circulating accelerators, cooling agents, adiaphoretics, purified water, etc.

In one aspect, the present disclosure provides a pharmaceutical composition for preventing or treating skin inflammation, including a complex extract prepared by mixing red ginseng, Centella asiatica, Houttuynia cordata, and Lonicerae Flos as active ingredients.

In an exemplary embodiment of the present disclosure, the skin inflammation may be at least one selected from the group consisting of atopic dermatitis, contact dermatitis, seborrheic dermatitis, and acne, but is not limited thereto.

The pharmaceutical composition of the present disclosure may further include an adjuvant in addition to the active ingredients. The adjuvant may be used with any adjuvant known in the art without limitation, but further include, for example, a Freund's complete adjuvant or incomplete adjuvant to increase the immunity thereof.

The pharmaceutical composition according to the present disclosure may be prepared in the form of incorporating the active ingredients into a pharmaceutically acceptable carrier. Here, the pharmaceutically acceptable carrier includes carriers, excipients and diluents, which are commonly used in a pharmaceutical field. The pharmaceutically acceptable carrier that may be used in the pharmaceutical composition of the present disclosure is not limited thereto, but may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oils.

The pharmaceutical composition of the present disclosure may be formulated and used in the form of oral formulations, such as powders, granules, tablets, capsules, suspensions, emulsions, syrups, aerosols, etc., external preparations, suppositories, or sterile injectable solutions according to each conventional method.

The formulations may be prepared by using diluents or excipients, such as a filler, an extender, a binder, a wetting agent, a disintegrating agent, a surfactant, etc., which are generally used. Solid formulations for oral administration include tablets, pills, powders, granules, capsules, etc., and these solid formulations may be prepared by mixing the active ingredients with at least one or more excipients, such as starch, calcium carbonate, sucrose, lactose, gelatin, etc. Further, lubricants such as magnesium stearate and talc may be used in addition to simple excipients. Liquid formulations for oral administration may correspond to suspensions, oral liquids, emulsions, syrups, etc., and may include various excipients, such as a wetting agent, a sweetener, an aromatic agent, a preserving agent, etc., in addition to commonly used diluents, such as water and liquid paraffin. Formulations for parenteral administration include sterile aqueous solutions, non-aqueous solvents, suspensions, emulsions, lyophilized agents, and suppositories. As the non-aqueous solvent and the suspension, propylene glycol, polyethylene glycol, vegetable oil such as olive oil, injectable ester such as ethyl oleate, etc. may be used. As a base material of the suppositories, witepsol, Tween 61, cacao butter, laurinum, glycerogelatin, etc. may be used.

The pharmaceutical composition according to the present disclosure may be administered to a subject through various routes. All methods of administration may be expected, and the pharmaceutical composition may be administered by, for example, oral, intravenous, intramuscular, subcutaneous, and intraperitoneal injection.

The dose of the pharmaceutical composition according to the present disclosure is selected in consideration of the age, body weight, sex, physical conditions, and the like of a subject. It is obvious that the concentration of the active ingredients included in the pharmaceutical composition may be variously selected according to a subject, and preferably, the active ingredients are included in the pharmaceutical composition at a concentration of 0.01 to 5,000 μg/ml. When the concentration thereof is less than 0.01 μg/ml, the pharmaceutical activity may not be exhibited, and when the concentration thereof is more than 5,000 μg/ml, the toxicity to the human body may be exhibited.

The pharmaceutical composition may be formulated into various oral or parenteral administration forms.

Formulations for oral administration include, for example, tablets, pills, hard and soft capsules, solutions, suspensions, emulsifiers, syrups, granules, etc., and these formulations may further include diluents (e.g., lactose, dextrose, sucrose, mannitol, sorbitol, cellulose and/or glycine), lubricants (e.g., silica, talc, stearic acid and its magnesium or calcium salts and/or polyethylene glycol), in addition to the active ingredients. In addition, the tablets may contain a binder such as magnesium aluminum silicate, starch paste, gelatin, tragacanth, methylcellulose, sodium carboxymethylcellulose and/or polyvinylpyrrolidine, and in some cases, may contain a disintegrant or effervescent mixture and/or absorbent such as starch, agar, alginate or sodium salts thereof, a coloring agent, a flavoring agent and a sweetening agent. The formulations may be prepared by conventional mixing, granulating or coating methods.

In addition, a typical formulation for parenteral administration is an injectable preparation, and a solvent for the injectable preparation may include water, a Ringer's solution, isotonic physiological saline or a suspension. Sterile fixed oils of the injection formulation may be used as a solvent or suspension medium, and any non-irritating fixed oil including mono- and di-glycerides may be used for this purpose.

In addition, the injectable preparation may use fatty acids such as oleic acid.

In one aspect, the present disclosure provides a method for preparing a complex extract including: a) preparing a mixture by mixing red ginseng, Centella asiatica, Houttuynia cordata, and Lonicerae Flos; and b) preparing a complex extract by ultrasonically extracting the mixture.

In an exemplary embodiment of the present disclosure, the yield of the complex extract prepared by the preparation method may be 5 to 15%, preferably 9 to 13%, more preferably 10 to 12%, and even more preferably 11.82%, but is not limited thereto.

In an exemplary embodiment of the present disclosure, the red ginseng, Centella asiatica, Houttuynia cordata, and Lonicerae Flos in step a) may be mixed in a weight ratio of 2 to 4:1 to 3:1 to 3:0.1 to 2, preferably 3:2:2:1, which is not limited thereto.

In an exemplary embodiment of the present disclosure, the ultrasonic extraction in step b) may be performed by using, as an extraction solvent, water, lower alcohols having 1 to 4 carbon atoms, or a mixed solvent thereof, preferably ethanol, more preferably 50 to 70% ethanol, and even more preferably 60% ethanol, but is not limited thereto.

In an exemplary embodiment of the present disclosure, the ultrasonic extraction in step b) may be performed at 30 to 70° C., preferably 40 to 60° C., more preferably 50° C., but is not limited thereto.

In an exemplary embodiment of the present disclosure, the ultrasonic extraction in step b) may be performed 1 to 3 times, preferably 2 times, but is not limited thereto.

As described above, the specific exemplary embodiments of the present disclosure have been described in detail, but those skilled in the art understanding the spirit of the present disclosure will be able to easily propose other degenerate inventions or other exemplary embodiments included in the scope of the present disclosure by adding, changing, and deleting other elements within the same technical scope. Therefore, it should be appreciated that the exemplary embodiments described above are illustrative in all aspects and are not restricted. The scope of the present disclosure is represented by claims to be described below rather than the detailed description, and it is to be interpreted that the meaning and scope of the claims and all the changes or modified forms derived from the equivalents thereof come within the scope of the present disclosure.

### <Example 1> Preparation of Complex Extract

To prepare a complex extract, 30 g of red ginseng, 20 g of Centella asiatica, 20 g of Houttuynia cordata, and 10 g of Lonicerae Flos were mixed with 500 mL of ethanol diluted to a concentration of 60%. Thereafter, the mixture was ultrasonically extracted twice at 50° C. every 2 hours, filtered through filter paper, and concentrated using a rotary vacuum evaporator. Thereafter, the mixture was freeze-dried and stored at −20° C. for use. The obtained sample was 9.48 g in total, showing a yield of 11.82%.

### <Experimental Example 1> Confirmation of Antioxidant Effect of Complex Extract

### 1-1. Electron Donating Ability Analysis

To confirm an antioxidant effect of the complex extract prepared in Example 1 above, electron donating ability was analyzed.

The electron donating ability referred to discoloration of purple to light yellow when exposed to an oxidizing substance due to a hydrogen donation effect by 1,1-diphenyl-2-picrylhydrazyl (DPPH), and the radical scavenging activity may be measured by measuring the absorbance.

Specifically, for electron donating ability assay, 50 μL of 0.2 mM 1,1-diphenyl-2-picrylhydrazyl (DPPH) was added to 100 μL of each sample solution, and the absorbance was measured at 517 nm after blocking light for 30 minutes at room temperature. The electron donating ability was expressed as a decrease ratio in absorbance between a sample solution-added group and a non-added group.

As a result, as shown in FIG. 1, when the concentration of the complex extract (DN) was 10, 50, 100, 500, and 1000 μg/mL, the activity was −7.48±3.04, 39.63±5.84, 56.47±5.11, 65.65±4.16, and 67.02±5.89%, respectively, indicating that the electron donating ability to DPPH tended to increase as the concentration of the complex extract increased.

As a result, it was confirmed that the complex extract prepared by mixing red ginseng, Centella asiatica, Houttuynia cordata, and Lonicerae Flos had an antioxidant effect in a concentration-dependent manner.

### 1-2. ABTS Radical Scavenging Activity Assay

To confirm an antioxidant effect of the complex extract prepared in Example 1 above, ABTS radical scavenging activity was analyzed.

The ABTS radical scavenging activity referred to discoloration of blue-green when exposed to an oxidizing substance, and may be measured by measuring absorbance.

Specifically, for ABTS radical scavenging activity assay, 7.4 mM 2,2-azino-bis(3-ethylbenzth iazoline-6-sulfonicacid) and 2.6 mM potassium persulfate were mixed in a 1:1 ratio and reacted in a dark room at room temperature for 24 hours, and then the mixture was diluted with water until the absorbance value at 734 nm was 0.700±0.001 and used. 100 μL of ABTS+solution was added to 100 μL of the sample solution, and the absorbance was measured at 734 nm after blocking light at room temperature for 1 minute. The radical scavenging activity was expressed as a decrease ratio in absorbance between the sample solution-added group and the non-added group.

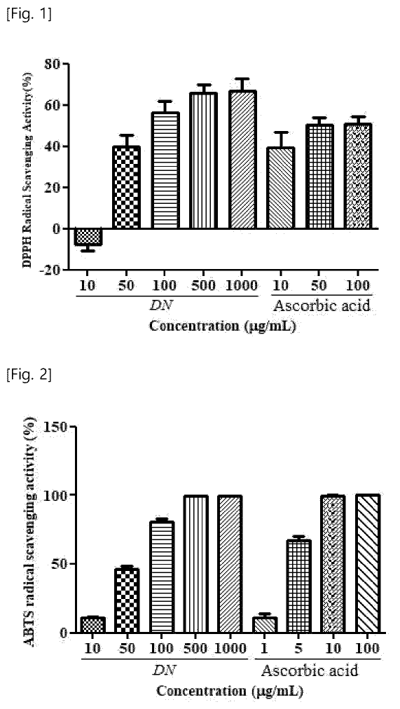

As a result, as shown in FIG. 2, when the concentration of the complex extract (DN) was 10, 50, 100, 500, and 1000 μg/mL, the activity was −0.95±0.63, 46.09±2.24, 80.66±2.11, 99.13±0.33, 99.16±0.64%, respectively, indicating that the ABTS radical scavenging activity tended to increase as the concentration of the complex extract increased.

As a result, it was confirmed that the complex extract prepared by mixing red ginseng, Centella asiatica, Houttuynia cordata, and Lonicerae Flos had an antioxidant effect in a concentration-dependent manner.

### 1-3. Reducing Power Assay

To confirm an antioxidant effect of the complex extract prepared in Example 1 above, reducing power was analyzed.

The reducing power referred to measuring the ability to donate electrons to reactive oxygen species and free radicals among various mechanisms of antioxidant activity, and the reducing power of a ferric-ferricyanide (Fe³⁺) mixture to be converted to ferrous (Fe²⁺) by donating hydrogen was expressed by measuring the absorbance at 700 nm.

Specifically, for reducing power assay, 0.3 mL of each concentration sample was mixed and prepared with 0.3 mL of 200 mM sodium phosphate buffer (pH 6.6) and 0.3 mL of 1% potassium ferricyanide. The mixture was reacted at 50° C. for 20 minutes, and then added with 0.3 mL of 10% trichloroacetic acid (TCA) to stop the reaction. Thereafter, the mixture was centrifuged at 12,000×g for 10 minutes, and 0.5 mL of the supernatant was mixed with 0.5 mL of deionized water and 0.1 mL of 0.1% ferric chloride and reacted for 10 minutes. The absorbance of the reaction solution was measured at 700 nm using an ELISA reader (PowerWave XS, Biotek, Winooski, VT, USA).

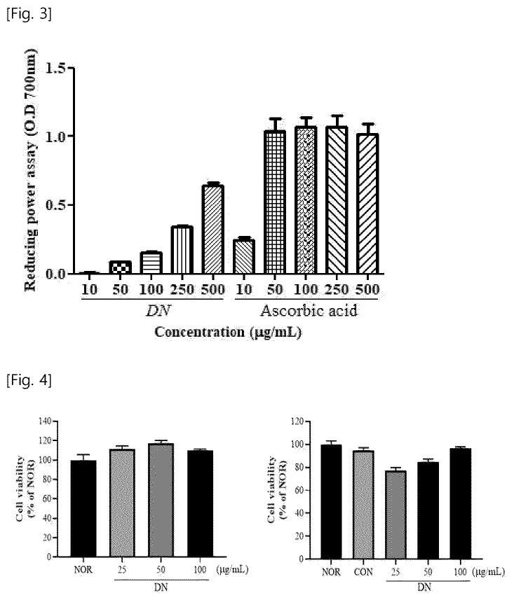

As a result, as shown in FIG. 3, when the concentration of the complex extract (DN) was 10, 50, 100, 250, and 500 μg/mL, the activity was 0.006±0.004, 0.09±0.004, 0.15=0.006, 0.34±0.007, 0.64±0.02, respectively, indicating that the reducing power tended to increase as the concentration of the complex extract increased.

As a result, it was confirmed that the complex extract prepared by mixing red ginseng, Centella asiatica, Houttuynia cordata, and Lonicerae Flos had an antioxidant effect in a concentration-dependent manner.

### 1-4. Analysis of Total Phenol and Total Flavonoid Contents

In order to confirm the antioxidant effect of the complex extract prepared in Example 1 above, the total phenol and total flavonoid contents were analyzed.

Specifically, in order to measure the total phenol content, 50 μL of each concentration sample was mixed with 50 μL of Folin-Ciocalteau reagent, and after 5 minutes, added with 50 μL of 10% Na₂CO₃ and then mixed by intermittent shaking, and the reaction mixture was reacted at room temperature for 60 minutes. The absorbance was measured at 640 nm and the data was calculated as mg of tannic acid equivalent (TAE) per g of the extract by diluting tannic acid to prepare a calibration curve.

In addition, to measure the total flavonoid content, 50 μL of the sample and quercetin were mixed with 200 μL of dimethylene glycol and 50 μL of 1 N NaOH. After reacting at 37° C. for 60 minutes, the absorbance of each reaction mixture was measured at 420 nm, and the data was calculated as mg of quercetin equivalent (QE) per g of the extract by diluting quercetin to prepare a calibration curve.

As a result, as shown in Table 1 below, the total phenol content was measured as 155.8 mg TAE/g and the total flavonoid content was measured as 185.7 mg QE/g at 100 g/mL of a complex extract concentration.

As a result, it was confirmed that the complex extract prepared by mixing red ginseng, Centella asiatica, Houttuynia cordata, and Lonicerae Flos had an antioxidant effect in a concentration-dependent manner.

TABLE 1

Complex extract (100 μg/mL)

| | | |
|---|---|---|
| Total phenol content (TPC) | 155.8 | mg TAE/g |
| Total flavonoid content (TFC) | 185.7 | mg QE/g |

### <Experimental Example 2> Confirmation of Cytotoxicity of Complex Extract

### 2-1. Cell Culture

Raw 264.7 cells, as mouse macrophages, were subcultured in a Dulbecco's Modified Eagle Medium (DMEM, Gibco, Grand Island, NY, USA) containing 100 units/mL penicillin-streptomycin and 10% fetal bovine serum (FBS) at 37° C. in a 5% CO₂ incubator.

### 2-2. Cell Viability Assay

In order to confirm the cytotoxicity of the complex extract prepared in Example 1 above, the viability of the cells incubated in Experimental Example 2-1 was analyzed.

Specifically, Raw 264.7 cells were inoculated and incubated in a 96-well plate at 1.5×10⁴ cells/well, and then the cells were pretreated with the complex extract at various concentrations of 25, 50, and 100 μg/mL, and after 1 hour, treated with LPS (1 μg/mL) and incubated for 24 hours. Thereafter, MTT was treated to each well and further incubated for 1 hour, and then the supernatant was removed, formazan was dissolved in DMSO, and the absorbance was measured at 540 nm. At this time, the cell viability was calculated as the relative cell viability (%) of the treated group based on the 100% viability of an NOR group that was not treated with the sample.

As a result, as shown in FIG. 4, it was confirmed that there was no cytotoxicity in both a case where the complex extract was treated alone and a case where the complex extract and LPS were treated together.

### <Experimental Example 3> Confirmation of Anti-Inflammatory Effect of Complex Extract

### 3-1. Analysis of Nitric Oxide (NO) Production Inhibitory Effect

To confirm an anti-inflammatory effect of the complex extract prepared in Example 1 above, a nitric oxide (NO) production inhibitory effect was analyzed.

NO was produced from L-arginine by NOS as a regulatory factor that played an important role in inflammatory responses, and when the inflammatory response occurred, a large amount of NO was produced in related cells, which was known to act as a cytotoxic substance and induced a pathological state, thereby causing genetic mutations and nerve and tissue damages.

Specifically, in order to analyze the nitric oxide (NO) production inhibitory effect, Raw 264.7 cells incubated in Experimental Example 2-1 above were inoculated and incubated in a 24-well plate at 8×10⁴ cells/well, and then pretreated with the extract at concentrations of 25, 50, and 100 μg/mL for 1 hour, treated with LPS, and incubated for 24 hours. Thereafter, 100 μL of the supernatant was placed in a 96-well plate, and mixed with 100 μL of a Griess reagent, and then the absorbance was measured at 540 nm. At this time, the NO production was calculated as a relative NO production (%) based on the NO production in a CON group treated only with LPS.

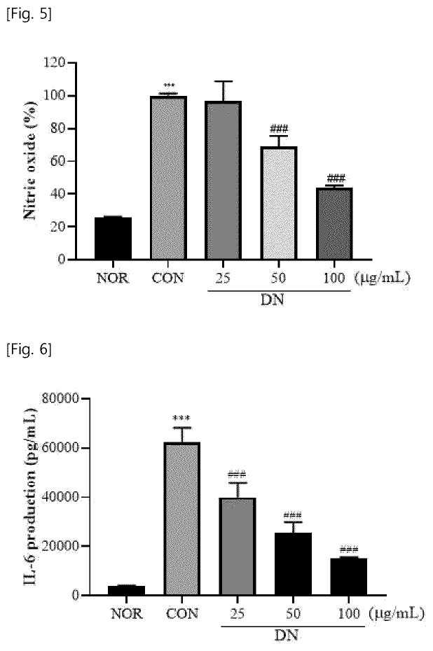

As a result, as shown in FIG. 5, the NO production was decreased in a concentration-dependent manner, as 26.0±0.3% in a NOR group, 100.0±1.3% in a LPS-treated CON group, and 96.7±12.1%, 69.1±6.4%, and 44.1±1.2% in experimental groups treated with 25, 50, and 100 μg/mL of the complex extract and LPS, respectively.

As a result, it was confirmed that the complex extract prepared by mixing red ginseng, Centella asiatica, Houttuynia cordata, and Lonicerae Flos had an anti-inflammatory effect by inhibiting the production of NO as an inflammatory factor.

### 3-2. Analysis of Inflammatory Cytokine Production Inhibitory Effect

To confirm an anti-inflammatory effect of the complex extract prepared in Example 1 above, an inflammatory cytokine production inhibitory effect was analyzed.

When an inflammatory response occurred, inflammatory cytokines such as TNF and IL-6 were secreted from immune cells. TNF was an important factor in normal immune responses, but caused immune-related diseases if produced excessively and induced the secretion of other inflammatory cytokines, and IL-6 was immediately produced and secreted for tissue damage or pathogens to mediate the induction of inflammation.

Specifically, in order to analyze the inflammatory cytokine production inhibitory effect, Raw 264.7 cells incubated in Experimental Example 2-1 above were inoculated and incubated in a 24-well plate at 8×10⁴ cells/well, and then pretreated with the extract at concentrations of 25, 50, and 100 μg/mL for 1 hour, treated with LPS, and incubated for 24 hours. Thereafter, the supernatant was centrifuged at 4,000 rpm for 5 minutes and then taken, and the proinflammatory cytokine production was measured using an ELISA kit according to a method suggested in the kit.

As shown in FIG. 6, as a result of measuring the IL-6 production, the IL-6 production was measured as 3824±269, 62461±5752, 40064±5784, 25757±4050, and 15089±521 μg/mL in the NOR group, CON group, DN 25 μg/mL treated group, DN 50 μg/mL treated group, and DN 100 μg/mL treated group, respectively, and it was confirmed that the IL-6 increased by LPS was decreased by the complex extract (DN) in the concentration-dependent manner.

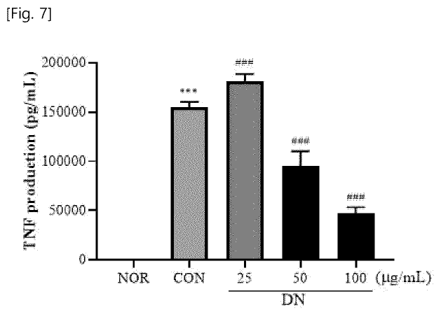

In addition, as shown in FIG. 7, as a result of measuring the TNF production, the TNF production was measured as 13.9±307.7, 154808±5361, 180611±7975, 94846±15583, and 47165±6,095 μg/mL in the NOR group, CON group, DN 25 μg/mL treated group, DN 50 μg/mL treated group, and DN 100 μg/mL treated group, respectively, and it was confirmed that the TNF increased by LPS was decreased by the complex extract (DN) in the concentration-dependent manner.

As a result, it was confirmed that the complex extract prepared by mixing red ginseng, Centella asiatica, Houttuynia cordata, and Lonicerae Flos had an anti-inflammatory effect by inhibiting the production of inflammatory cytokines TNF and IL-6.

In other words, the complex extract prepared by mixing red ginseng, Centella asiatica, Houttuynia cordata, and Lonicerae Flos has excellent antioxidant and anti-inflammatory effects, and thus may be effectively used as a composition for enhancing a skin barrier or a composition for improving skin inflammation.

Hereinabove, the present disclosure has been described with reference to preferred Examples and Experimental Examples thereof. It will be understood to those skilled in the art that the present disclosure may be implemented as modified forms without departing from an essential characteristic of the present disclosure. Therefore, the disclosed embodiments should be considered in an illustrative viewpoint rather than a restrictive viewpoint. The scope of the present disclosure is illustrated by the appended claims rather than by the foregoing description, and all differences within the scope of equivalents thereof should be construed as being included in the present disclosure.

What is claimed is:

1. A method for preparing a complex extract comprising:
a) preparing a mixture by mixing red ginseng, Centella asiatica, Houttuynia cordata and Lonicerae Flos; and
b) preparing a complex extract by ultrasonically extracting the mixture.

2. The method for preparing the complex extract of claim 1, wherein the yield of the complex extract prepared by the preparation method is 5 to 15%.

3. A method for preventing or treating skin inflammation, the method comprising administering to a subject in need thereof the complex extract prepared by the method of claim 1 as active ingredients.

4. The method according to claim 3, wherein the skin inflammation is at least one selected from the group consisting of atopic dermatitis, contact dermatitis, seborrheic dermatitis, and acne.

5. A method for enhancing a skin carrier, the method comprising administering to a subject in need thereof the complex extract prepared by the method of claim 1 as active ingredients.

6. The method according to claim 5, wherein the complex extract is prepared by mixing red ginseng, Centella asiatica, Houttuynia cordata, and Lonicerae Flos in a weight ratio of 2 to 4:1 to 3:1 to 3:0.1 to 2.

7. The method according to claim 5, wherein the complex extract is extracted with water, lower alcohols having 1 to 4 carbon atoms, or a mixed solvent thereof.

8. The method according to claim 5, wherein the complex extract is prepared by ultrasonic extraction at 30 to 70° C.

9. The method according to claim 8, wherein the ultrasonic extraction is performed 1 to 3 times.

10. The method according to claim 5, wherein the subject requires an antioxidant effect.

11. The method according to claim 5, wherein the subject requires an anti-inflammatory effect.
